(19) United States
(12) Patent Application Publication
(10) Pub. No.: US 2004/0142049 A1
(43) Pub. Date: Jul. 22, 2004

(54) Compositions having tnf production inhibitory effect and tnf production inhibitors

(76) Inventors: Tatsumasa Mae, Kakogawa-shi (JP); Misuzu Tsukagawa, Akashi-shi (JP); Mikio Kitahara, Kobe-shi (JP); Kaku Nakagawa, Kyoto-shi (JP)

Correspondence Address: Kenyon & Kenyon, Suite 700, 1500 K Street NW, Washington, DC, 20005-1257, US

(21) Appl. No.: 10/480,930

(22) PCT Filed: Jul. 17, 2002
(86) PCT No.: PCT/JP02/07244

(30) Foreign Application Priority Data
Jul. 17, 2001 (JP) 2001-216171

(51) Int. Cl. A61K 35/78

(52) U.S. Cl. 424/739; 424/756; 424/757; 424/778

(57) ABSTRACT

The object of the present invention is to provide a composition having a TNF production inhibitory effect and TNF production inhibitors, which raise no adverse effect or safety problem, can inhibit any excessive production of TNF and is useful in the prevention or amelioration of various TNF-mediated diseases, such as chronic inflammatory diseases, acute inflammatory diseases, infectious inflammatory diseases, autoimmune diseases, and allergic diseases.

It is found out that cinnamon extracts, clove extracts, licorice root extracts, and ginger extracts each has a TNF production inhibitory effect. Thus, the above object can be achieved by compositions having a TNF production inhibitory effect and TNF production inhibitors which contain at least one of cinnamon extracts, clove extracts, licorice root extracts, and ginger extracts.

### TECHNICAL FIELD

The present invention relates to a composition having a TNF production inhibitory effect and to TNF production inhibitors. More particularly, it relates to a composition having a TNF production inhibitory effect and TNF production inhibitors, which are useful in the prevention or amelioration of various TNF-mediated diseases, such as chronic inflammatory diseases, acute inflammatory diseases, infectious inflammatory diseases, autoimmune diseases, and allergic diseases, by inhibiting an excessive production of TNF.

### BACKGROUND ART

TNF (Tumor Necrosis Factor) was first discovered as an antitumor substance and, later, its character as a cytokine involved in inflammation was revealed. TNF includes TNF-α and TNF-β (lymphotoxin). It is known that TNF-α is produced in response to various stimuli provided by a variety of cells, typically macrophages and monocytic cells and TNF-β is produced by T cells. As long as TNF is produced in vivo at normal levels, it plays an important role in defending living organisms, improving their immunocompetence, for instance. However, when TNF is produced and secreted in excess by some or other cause, it causes morbid inflammation, inducing or promoting TNF-mediated diseases such as chromic inflammatory diseases, acute inflammatory diseases, infectious inflammatory diseases, autoimmune diseases, and allergic diseases. Therefore, expectedly, such TNF-mediated diseases may be prevented or ameliorated by inhibiting the excessive production of TNF.

In Japanese Kokai Publication Hei-07-215884, there is disclosed a perilla extract having a TNF production inhibitory effect as prepared by grinding the foliage of a plant of the family Labiatae, extracting the ground foliage with water, an organic solvent such as ethanol, or a mixture thereof, depriving the extract composition of perillaldehyde and a fraction exceeding 10,000 in molecular weight from the extract. In that document, it is also disclosed that this perilla extract is effective against allergic diseases such as atopic dermatitis.

In Japanese Kokai Publication Hei-03-157330, it is disclosed that epigallocatechin gallate, which is a component of a leaf of tea (Camellia sinensis L.), is effective as antiallergic agent. In Japanese Kokai Publication Hei-10-72361, it is disclosed that a tea leaf extract obtained by extracting tea leaves with water, an organic solvent, or a mixture thereof has a TNF production inhibitory effect.

In addition to such perilla extract and tea leaf extract, curcumin, which is a yellow color component of turmeric (Curcuma longa L.) of the family Zingiberaceae, has a TNF production inhibitory effect, as reported by Marion Man-Ying Chan (Biochemical Pharmacology, 49, 1551-1556, 1995) and by Yoshiaki Abe et al. (Pharmacological Research, 39, 41-47, 1999). It is unknown, however, as to whether cinnamon, clove, licorice root, or ginger, or an extract thereof has a TNF production inhibitory effect.

### SUMMARY OF THE INVENTION

In view of the foregoing, it is an object of the present invention to provide a composition having a TNF production inhibitory effect and TNF production inhibitors, which raise no adverse effect or safety problem, can inhibit any excessive production of TNF and are useful in the prevention or amelioration of TNF-mediated diseases.

The present inventors made intensive investigations to accomplish the above object and, as a result, found that cinnamon extracts, clove extracts, licorice root extracts, and ginger extracts each has a TNF production inhibitory effect. Based on this finding, they have now completed the present invention.

The present invention thus relates to a composition having a TNF production inhibitory effect,

which comprises at least one of cinnamon extracts, clove extracts, licorice root extracts, and ginger extracts.

Further, the invention relates to a TNF production inhibitor,

which comprises at least one of cinnamon extracts, clove extracts, licorice root extracts, and ginger extracts.

Furthermore, the invention relates to the above TNF production inhibitor,

which is intended for food/beverage use; and the above TNF production inhibitor,

which is intended for pharmaceutical use.

The invention also relates to an agent for the prevention or amelioration of TNF-mediated diseases,

which comprises the above TNF production inhibitor.

### DETAILED DISCLOSURE OF THE INVENTION

In the following, the present invention is described in detail.

The composition having a TNF production inhibitory effect and the TNF production inhibitors according to the invention each contains at least one of cinnamon extracts, clove extracts, licorice root extracts, and ginger extracts. The composition having a TNF production inhibitory effect can be used in the form of preparations such as foods and drugs, while the TNF production inhibitors can be used in the form of such an extract itself or in the form of preparations derived therefrom.

The starting plant cinnamon to be used in the practice of the invention is Cinnamomum cassia, C. zeylanicum or C. loureirii of the family Lauraceae; the plant clove is Syzygium aromaticum or Eugenia caryophyllata of the family Myrtaceae; the plant licorice root is Glycyrrhiza glabra, G. uralensis or G. inflate of the family Fabaceae; and the plant ginger is Zingiber officinale of the family Zingiberaceae. These all have long and wide experience as food ingredients or spices and extracts thereof have been approved as food additives. Thus, they present no adverse effect or safety problem.

The cinnamon extracts, clove extracts, licorice root extracts, and ginger extracts to be used in the practice of the invention can be obtained from the plants mentioned above by solvent extraction, among others. The method of obtaining the extracts is not limited to solvent extraction but other extraction procedures such as steam distillation and extraction with carbon dioxide according to the supercritical extraction technology. Furthermore, the extracts may be used in the practice of the invention in the form of crude extracts or half-purified extracts unless they contain impurities rendering them inappropriate for use in foods, drinks or drugs.

In carrying out the solvent extraction, each of the above plants, in a powder, ground or original form, is, for example, steeped in 1 to 20 volumes of such a solvent as mentioned below, and the mixture is stirred or allowed to stand at −20 to 100° C., preferably 1 to 80° C., more preferably 20 to 60° C., for 0.1 hour to 1 month, preferably 0.5 hour to 7 days. Then, the mixture is filtered or centrifuged, and the extract solution obtained is concentrated to remove the solvent to give the desired extract.

The solvent to be used for extraction includes, among others, water, acetone, ethanol, glycerol, ethyl acetate, propylene glycol, hexane, and cooking fats and oils. Two or more of these solvents may also be used in admixture. Preferably used are organic solvents readily removal after extraction, for example acetone, ethanol, ethyl acetate, and hexane.

The thus-obtained cinnamon extracts, clove extracts, licorice root extracts, and ginger extracts each contains a component having a TNF production inhibitory effect. Further, such component having a TNF production inhibitory effect may be used in a form concentrated or isolated from those extracts.

The method of evaluating the above extracts for a TNF production inhibitory effect is not particularly restricted but the above extracts can be evaluated, for example, by adding or administering them to an experimental system in which the production of TNF is inducible. Thus, the above extracts can be evaluated in vitro by stimulating human cells, for example monocytes and other monocytic cells, with PMA (phorbol 12-myristyl 13-acetate) or LPS (lipopolysaccharide), for instance, for inducing the production of TNF-α, adding any of the above extracts thereto, cultivating the cells, and determining the TNF-α concentration in the culture medium. The evaluation can be made also in vivo by using mice administered with LPS or the immunostimulant romurtide or OK432 or actually using allergic disease model mice, administering any of the above extracts and, thereafter, determining the TNF-α concentration in blood.

The composition having a TNF production inhibitory effect and the TNF production inhibitors, both according to the invention, can each be used for a food/beverage and a drug. Those are not restricted in form but they may be used, for example, in the form of a food/beverage, such as food with health claims (food for specified health uses and food with nutrient function claims) or health food, or a drug, or a quasi drug, for instance.

For use as a food/beverage, it can be directly ingested or may be formulated into easily ingestable products, such as capsules, tablets, granules, and the like, with the aid of a known carrier, auxiliary agent or the like for ingestion. The amount of the TNF production inhibitors of the invention in such a formulated product is preferably 0.1 to 100 weight %, more preferably 10 to 90 weight %. Furthermore, it can be mixed into raw materials for all kinds of food and beverage products, for example confections such as chewing gum, chocolate, candies, jellies, biscuits, crackers, etc.; frozen sweets such as ice cream, ice candies, etc.; beverages such as tea, nonalcoholic beverages, nutritional drinks, drinks for beauty, etc.; noodles such as Japanese wheat noodles, Chinese noodles, spaghetti, instant noodles, etc.; fish paste foods such as fish minced and steamed (kamaboko), fish sausage (chikuwa), minced flesh (hannpen), etc.; seasonings such as dressings, mayonnaise, sauces, etc.; oleaginous products such as margarine, butter, salad oil, etc.; bakery products, hams, soups, retort foods, frozen foods, and so forth. In taking such a food or beverage TNF production inhibitors, the daily intake for an adult human is preferably 0.1 to 1,000 mg/kg body weight, more preferably 1 to 100 mg/kg body weight, on an extract content basis.

For use as a pharmaceutical product, the dosage form is not particularly restricted but includes capsules, tablets, granules, injections, suppositories, and patches. Such dosage forms can be prepared by suitably formulating pharmaceutically acceptable material for preparation such as excipient, disintegrator, lubricant, binder, antioxidant, coloring agent, aggregation inhibitor, absorption promoter, solubilizing agent, stabilizer, and so on. The daily dosage of such a preparation for adult human is preferably 0.1 to 1,000 mg/kg, more preferably 1 to 100 mg/kg, on an extract content basis, which dosage is to be administered once a day or in a few divided doses a day.

For use as a quasi-drug, the composition can be used in such forms as, inter alia, ointments, liniments, aerosols, creams, soaps, face cleansers, body cleansers, toilet water, lotions, and bath agents, with an additive or the like when necessary, which forms enable a use to body parts.

An agent for the prevention or amelioration of TNF-mediated diseases can be obtained using the composition having a TNF production inhibitory effect or the TNF production inhibitors according to the invention. Since the cinnamon extract, clove extract, licorice root extract, and/or ginger extract contained in the TNF production inhibitors each can inhibit the production of TNF, the agent is useful in the prevention or amelioration of various TNF-mediated diseases such as chronic inflammatory diseases, acute inflammatory disease, infectious inflammatory diseases, autoimmune diseases, and allergic diseases.

By chronic inflammatory diseases are meant herein, among others, osteoarthritis, psoriatic arthritis, inflammatory skin diseases (psoriasis, eczematous dermatitis, seborrheic dermatitis, lichen planus, pemphigus, bullous pemphigoid, bullous epidermolysis, urticaria, vascular edema, vasculitis, erythema, dermal eosinophilia, acne, alopecia areata, eosinophilic fasciitis, atherosclerosis, etc.), inflammatory intestinal diseases (ulcerative colitis, Crohn's disease, etc.), and like diseases.

By acute inflammatory diseases are meant, among others, contact dermatitis, adult respiratory distress syndrome (ARDS), septicemia (inclusive of septicemia-induced organopathy etc.), septic shock, and like diseases.

By infectious inflammatory diseases are meant, among others, endotoxin shock, acquired immunodeficiency syndrome (AIDS), cachexia and, further, inflammatory reactions caused by infection with bacteria, viruses, mycoplasma, etc. (inclusive of fever, pain, organopathy, etc. due to enzootic fever or nonenzootic fever), and like diseases.

By autoimmune diseases are meant, among others, rheumatoid arthritis, ankylosing spondylitis, systemic lupus erythematosus, glomerulonephritis (e.g. nephrotic syndrome (e.g. idiopathic nephrotic syndrome, minimal change nephropathy)), multiple sclerosis, multiple chondritis, scleroderma, dermatomyositis, Wegener's granulomatosis, active chronic hepatitis, primary biliary cirrhosis, myasthenia gravis, idiopathic sprue, Grave's disease, sarcoidosis, Reiter's syndrome, juvenile diabetes (type I diabetes mellitus), autoimmune ophthalmic diseases (endocrine ophthalmopathy, noninfectious uveitis, keratitis (e.g. keratoconjunctivitis sicca, vernal keratoconjunctivitis)), autoimmune blood diseases (hemolytic anemia, aplastic anemia, idiopathic thrombocytopenia, etc.), and like diseases.

By allergic diseases are meant, among others, atopic dermatitis, asthmatic diseases (bronchial asthma, infantile asthma, allergic asthma, intrinsic asthma, extrinsic asthma, dust asthma, delayed asthma, respiratory tract hypersensitivity, bronchitis, etc.), allergic rhinitis, and like diseases.

As other TNF-mediated diseases, there may be mentioned, among others, insulin-resistant type II diabetes, resistive reactions, namely rejections and graft versus host (GvH) diseases, upon transplantation of organs or tissues (homologous or heterologous transplantation of heart, kidney, liver, lung, bone marrow, cornea, pancreas, islet cells, small intestine, duodenum, extremities, muscle, nerve, fatty marrow, skin, etc.), osteoporosis, cancerous cachexia, disseminated intravascular coagulation, trauma, burn, inflammatory reactions (inclusive of shock) induced, for example, by animal or plant components (inclusive of venom etc.) or due to drug administration, and like diseases.

### BEST MODE FOR CARRYING OUT THE INVENTION

The following examples illustrate the present invention more specifically. These examples are, however, by no means limitative of the scope of the invention.

### EXAMPLE 1

### Preparation of a Cinnamon Extract

Using a glass vessel, 1,000 g of cinnamon powder (Kaneka Sun Spice Co., Ltd.) was steeped in 5 volumes of ethyl acetate and allowed to stand at room temperature, protected against light, for 1 week with occasional stirring. The mixture was then filtered through filter paper (ADVANTEC No. 2) twice to remove the powder and recover an extract solution. This extract solution was concentrated under reduced pressure to remove the solvent and recover 59.57 g of a cinnamon extract.

### EXAMPLE 2

### Preparation of a Clove Extract

Using a glass vessel, 600 g of clove powder (Kaneka Sun Spice Co., Ltd.) was steeped in 5 volumes of ethyl acetate and allowed to stand at room temperature, protected against light, for 1 week with occasional stirring. The mixture was then filtered through filter paper (ADVANTEC No. 2) twice to remove the powder and recover an extract solution. This extract solution was concentrated under reduced pressure to remove the solvent and recover 47.59 g of a clove extract.

### EXAMPLE 3

### Preparation of a Licorice Extract

Using a glass vessel, 500 g of licorice powder (Kaneka Sun Spice Co., Ltd.) was steeped in 5 volumes of ethyl acetate and allowed to stand at room temperature, protected against light, for 1 week with occasional stirring. The mixture was filtered through filter paper (ADVANTEC No.2) twice to remove the powder and recover an extract solution. The extract solution was concentrated under reduced pressure to remove the solvent, whereupon 33.91 g of a licorice extract was obtained.

### EXAMPLE 4

### Preparation of a Ginger Extract

Using a glass vessel, 700 g of ginger powder (Kaneka Sun Spice Co., Ltd.) was steeped in 5 volumes of ethanol and allowed to stand at room temperature, protected against light, for 1 week with occasional stirring. The mixture was filtered through filter paper (ADVANTEC No.2) twice to remove the powder and recover an extract solution. The extract solution was concentrated under reduced pressure to remove the solvent, whereupon 41.03 g of a ginger extract was obtained.

### EXAMPLE 5

### TNF Production Inhibitory Effect

Blood (50 ml; heparin-added) was collected from each healthy volunteer, and mononuclear cells were isolated using Ficoll-Paque PULS (Amersham Pharmacia Biotech). The mononuclear cells obtained were washed with PBS (phosphate buffered physiological saline) three times, then suspended in RPMI 1640 medium (Life Technologies) to a concentration of 5×10⁶ cells/ml, and sowed onto a 96-well culture plate at a rate of 160 μl/well (=8×10⁵ cells/well). After 1 hour of incubation in a 5% CO₂ incubator at 37° C., the cells not adhering to the plate were removed by washing with PBS. RPMI 1640 medium containing 10% FBS (fetal bovine serum) with the extract (1 to 30 μg/ml) obtained in each of Examples 1 to 4 and PMA (phorbol 12-myristyl 13-acetate, 15 ng/ml) added was added to each well in an amount of 150 μl/well, followed by 18 to 20 hours of incubation in a 5% CO₂ incubator at 37° C. Then, the human TNF-α concentration in the medium was determined using an ELISA kit (Life Technologies). Further, viable cells were counted using Cell Counting Kit-8 (DOJINDO LABORATORIES).

The TNF-α levels and viable cells counts thus found are shown in Table 1 in terms of percentages (% control) relative to the respective values obtained in the control group (no extract added, PMA alone added) which are each taken as 100%.

TABLE 1

| | | Amount of | Viable cell |
|---|---|---|---|
| | Addition | TNF-α | count |
| | level | (% control) | (% control) |
| Control | — | 100 | 100 |
| Cinnamon | 10 μg/ml | 78 ± 24 | 106 |
| extract | 30 μg/ml | 8 ± 5 | 116 |
| Clove | 1 μg/ml | 69 ± 5 | 82 |
| extract | 3 μg/ml | 46 ± 2 | 117 |
| | 10 μg/ml | 21 ± 7 | 109 |
| | 30 μg/ml | 9 ± 6 | 120 |
| Licorice | 1 μg/ml | 112 ± 3 | 79 |
| extract | 3 μg/ml | 75 ± 2 | 122 |
| | 10 μg/ml | 23 ± 13 | 108 |
| | 30 μg/ml | 4 ± 3 | 114 |
| Ginger | 1 μg/ml | 83 ± 2 | 116 |
| extract | 3 μg/ml | 78 ± 2 | 128 |
| | 10 μg/ml | 49 ± 5 | 121 |
| | 30 μg/ml | 8 ± 8 | 107 |

As is evident from Table 1, the cinnamon extract, clove extract, licorice root extract, and ginger extract all reduced the TNF-α level in an addition level-dependent manner. With all the extracts, the viable cell counts were 79 to 128% relative to that in the control group, indicating that the decreases in TNF-α content were not due to cell deaths. These results proved that the cinnamon extract, clove extract, licorice root extract, and ginger extract all have a TNF production inhibitory effect.

### EXAMPLE 6

### Preparation of Cinnamon Extract-Containing

| tablets | |
|---|---|
| Cinnamon extract | 45 weight parts |
| Lactose | 35 weight parts |
| Crystalline cellulose | 15 weight parts |
| Sucrose fatty acid ester | 5 weight parts |

According to the above recipe, cinnamon extract-containing tablets for food/beverage use were manufactured by the established procedure.

### EXAMPLE 7

### Preparation of Clove Extract-Containing Soft

| capsules | |
|---|---|
| Clove extract | 40 weight parts |
| Sesame oil | 55 weight parts |
| Glycerin fatty acid ester | 5 weight parts |

According to the above recipe, clove extract-containing soft capsules for food/beverage use were manufactured by the established procedure.

### EXAMPLE 8

### Preparation of Licorice Extract-Containing

| Japanese wheat noodles | |
|---|---|
| Licorice extract | 1 weight part |
| Hard flour | 100 weight parts |
| Soft flour | 100 weight parts |
| Common salt | 10 weight parts |
| Water | 100 weight parts |

According to the above recipe, licorice extract-containing Japanese wheat noodles were manufactured by the established procedure.

### EXAMPLE 9

### Preparation of Ginger Extract-Containing Crackers

| | |
|---|---|
| Ginger extract | 1 weight part |
| Soft flour | 120 weight parts |
| Common salt | 1 weight part |
| Baking powder | 2 weight parts |
| Butter | 30 weight parts |
| Water | 40 weight parts |

According to the above recipe, ginger extract-containing crackers were manufactured by the established procedure.

### Industrial Applicability

According to the invention, there is provided a composition having a TNF production inhibitory effect as well as TNF production inhibitors. The composition having TNF inhibitory effect and the TNF inhibitor according to the invention are useful in the prevention or amelioration of various TNF-mediated diseases, such as chronic inflammatory diseases, acute inflammatory diseases, infectious inflammatory diseases, autoimmune diseases, and allergic diseases.

1. A composition having a TNF production inhibitory effect,
which comprises at least one of cinnamon extracts, clove extracts, licorice root extracts, and ginger extracts.

2. A TNF production inhibitor,
which comprises at least one of cinnamon extracts, clove extracts, licorice root extracts, and ginger extracts.

3. The TNF production inhibitor according to claim 2,
which is intended for food/beverage use.

4. The TNF production inhibitor according to claim 2,
which is intended for pharmaceutical use.

5. An agent for the prevention or amelioration of TNF-mediated diseases,
which comprises the TNF production inhibitor according to claim 2.
